(19) United States
(12) Patent Application Publication
(10) Pub. No.: US 2006/0175554 A1
(43) Pub. Date: Aug. 10, 2006

(54) Germicidal brush cleaner

(76) Inventor: Robert H. Riddell, Bellevue, WA (US)

Correspondence Address: DEAN A. CRAINE, SUITE 140, 400 - 112TH AVENUE NE, BELLEVUE, WA, 98004, US

(21) Appl. No.: 11/053,089

(22) Filed: Feb. 7, 2005

(51) Int. Cl. A61L 2/10

(52) U.S. Cl. 250/455.11

(57) ABSTRACT

A germicidal brush cleaner that uses a germicidal light source to disinfect the individual bristles on a plurality of toothbrushes and a hairbrush. Each toothbrush and hairbrush includes bristles made of optical fibers capable of transmitting ultraviolet light. There is also a special method of attachment of the toothbrush inside the cleaner that secures the toothbrushes in a set position in the holder. The germicidal light source may be a germicidal fluorescent ultraviolet lamp. The light rays from the germicidal light source are directed at the opposite ends of the bristle at the critical angle, or slightly greater than the critical angle, in order to attain total internal reflection of the light down the bristles of the toothbrushes.

### BACKGROUND OF THE INVENTION

1. Field of the Invention

This invention relates to devices used to sanitize brushes.

2. Description of the Related Art

Typically, optical fibers are used in communication cables, which use refractive index, critical angle, and total internal reflection to transmit light signals hundreds of miles. In order to go long distances without a loss of signal, they use coherent infrared light with boosters every so often, and cladding surrounding the core to prevent loss of signal.

The speed of light in the air is approximately 300,000 m/sec, while the speed of light in an optical fiber is slower, approximately 181,000 m/sec. Whenever light passes from one medium to another medium that has a lower speed of light, there will be a change in the direction of light. This change in direction is called the angle of refraction, and it always bends toward the normal. Normal is the line perpendicular to the interface between the air and the optical fiber. See Tables 1 and 2.

There is always a partial reflection of the light up until the critical angle of approximately 43 degrees, after which light is totally reflected. Therefore when light in air meets an optical fiber at an angle of incidence greater than the critical angle, the light will be totally reflected. This is the reason that a light signal can be transmitted for miles down an optical fiber with out much loss of the signal. The diagrams show these angles and how the light passes down an optical fiber.

### SUMMARY OF THE INVENTION

It is an object of the present invention to provide a germicidal brush cleaner.

It is a further object of this invention to provide such a cleaner that sterilizes toothbrushes and hairbrushes using coherent ultraviolet germicidal light.

These and other objects are met by the cleaner disclosed herein designed to clean both toothbrushes and hair brushes that have bristles made of optical fibers. The optical fibers which function like bristles on a standard toothbrush or hair brush, are designed to transmit ultraviolet light

The cleaner device includes an outer housing with a coherent, germicidal, ultraviolet light source located therein that is used to sanitize the bristles and the brush heads. The outer housing includes a central support member and two pivotally mounted doors that swing open along the rear end of the support member so that the opposite sides of the support member may be fully exposed. The opposite inside surfaces of the support member are concave. Mounted on the inside surface of each door is a concave reflector.

Mounted longitudinally on the inside surface of one door is a plurality of toothbrushes. Mounted on the concave surface of the support member opposite the surface adjacent to the toothbrushes is a longitudinally aligned hairbrush.

The bristles on the toothbrushes and hairbrush are fiber optics designed to transmit ultraviolet light. The toothbrushes are mounted on spring clamp retainers that allow the position of the toothbrushes to be adjusted so that the ends of the bristles may be positioned on the support surface so that the angle of incidence for the ultraviolet light is greater than the critical angle for total internal reflection of the light. Because the bristles are not covered with a cladding, some germicidal light is able to escape through the sides of the bristles and sanitizes the exterior surfaces of the adjacent bristles, and the surrounding areas on the toothbrush head.

Mounted on the front-end surface of the support member opposite the door hinges is an elongated germicidal light source. The germicidal light source is centrally mounted on the front-end surface of the support member and designed to transmit germicidal light on opposite directions toward the inside cavities formed in the device when the two doors are closed.

In the preferred embodiment, the hairbrush is vertically aligned and placed parallel to the concave inside surface of the support member on the side of the support member opposite the toothbrushes. The end of the hairbrush is mounted on a rotating vertical axle that extends upward from a short support arm that extends laterally from the base of the support member. The vertical axle is connected to a gearbox linked to an electric motor located inside the support member. When the electric motor is activated, the hairbrush rotates approximately 1 revolution every 15 minutes so that all of the bristles on the hairbrush are exposed to the ultraviolet light.

Because the approximate speed of light in the optical fiber can be determined, the critical angle for total internal reflection of the light in the bristle of the toothbrush can easily be set by moving the light source to different positions in the cleaner. Once the position of the germicidal light source is determined, further adjustment can be made to the position of the head and bristles by adjusting the spring clamp retainers that hold the heads of the toothbrushes. When all the adjustments are completed, the source of light and the heads of the toothbrushes are locked into position

### DESCRIPTION OF THE DRAWINGS

FIG. 1 is a top view of a perspective view of the germicidal brush cleaner with four toothbrushes mounted on the inside surface of one door and a circular hairbrush mounted on the inside surface of the support member opposite the toothbrushes.

FIG. 2 is a side elevational view of the cleaner showing both doors closed over the center support structure with four of toothbrushes and one circular hairbrush located therein.

FIG. 3 is a side elevational view of the center support structure.

FIG. 4 is a top plan view of the brush cleaner showing the two doors in an open position with four toothbrushes mounted on one door and the circular hairbrush attached to the vertical axle.

FIG. 5 is a top view showing a toothbrush head being held on the inside surface of the door by a spring clamp retainer.

FIG. 6 is a rear elevational view of the toothbrush mounted in a spring clamp retainer shown in FIG. 5.

FIG. 7 is a side elevational view of one optical toothbrush mounted on the inside surface of the door closed over the support member.

FIG. 8 is a side elevational view of the circular hairbrush mounted on the vertical axle that is attached to a short support arm that extends laterally from the support member.

### DESCRIPTION OF THE PREFERRED EMBODIMENT(S)

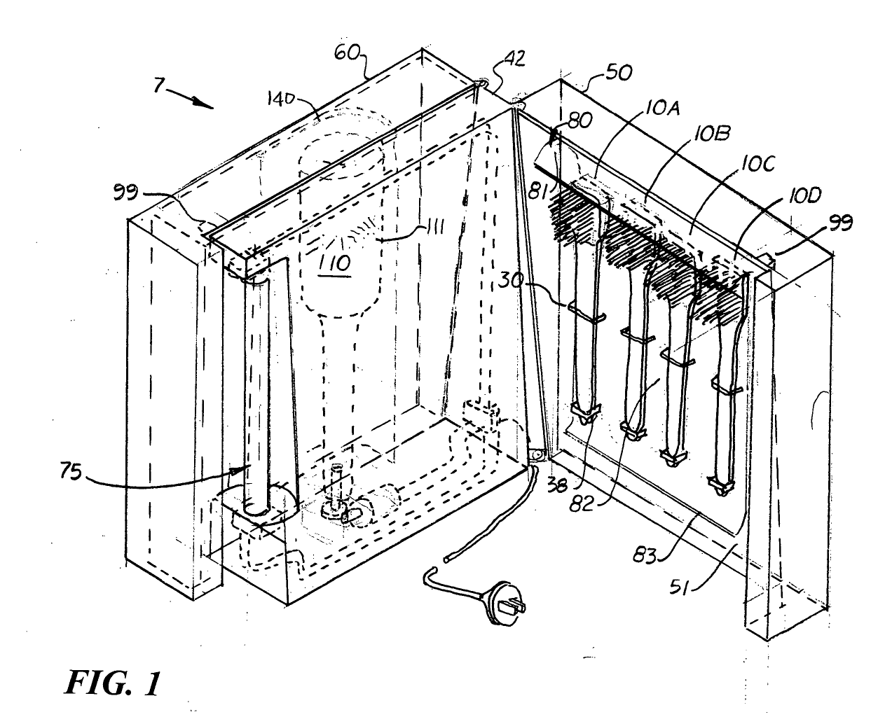

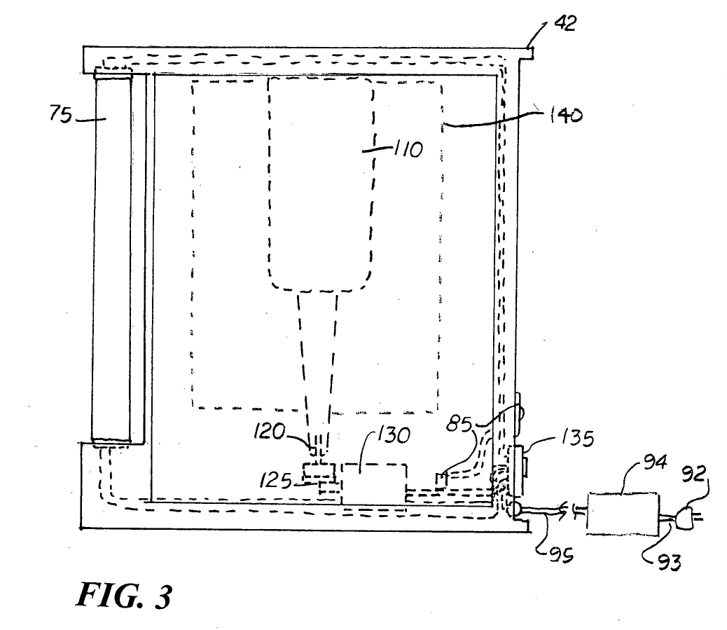

Referring to FIGS. 1-8, there is shown a germicidal brush cleaner 7 specifically design to sanitize a plurality of toothbrushes 10A-D and at least one hairbrush. The cleaner 7 uses an ultraviolet light source 75 to sanitize the bristles 20 on a plurality of toothbrushes 10A-D and on a circular hairbrush 110.

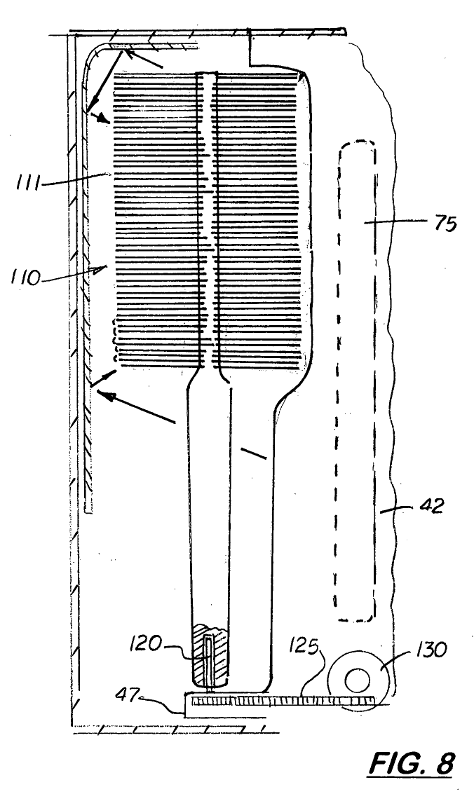

In the inventor's previous filed U.S. patent application (Ser. No. 10/865,8767, filed on Jan. 14, 2004), a germicidal toothbrush 10 is disclosed and now disclosed herein and shown in FIG. 8 that includes an elongated handle 12 about the same size and shape as a standard toothbrush with a head 16 located at one end with perpendicularly aligned bristles 20 attached thereto. The bristles 20 extend completely through the head 16, so that the distal and proximal ends 20A, 20B are exposed to light. Each bristle 20 is a single optical fiber capable of transmitting light from one end to the other. Between the end of the handle 12 and the head 16 is a narrow neck area 18, which is engaged by a spring clamp retainer 30 used to securely hold the toothbrush 10 in a set position in the cleaner 7.

The cleaner 7 is specifically designed not only to clean a plurality of germicidal toothbrushes designated 10A-D, but also a germicidal hairbrush 110 also including a plurality of bristles 111 also made of optic filer capable of transmitting light from one end to the other.

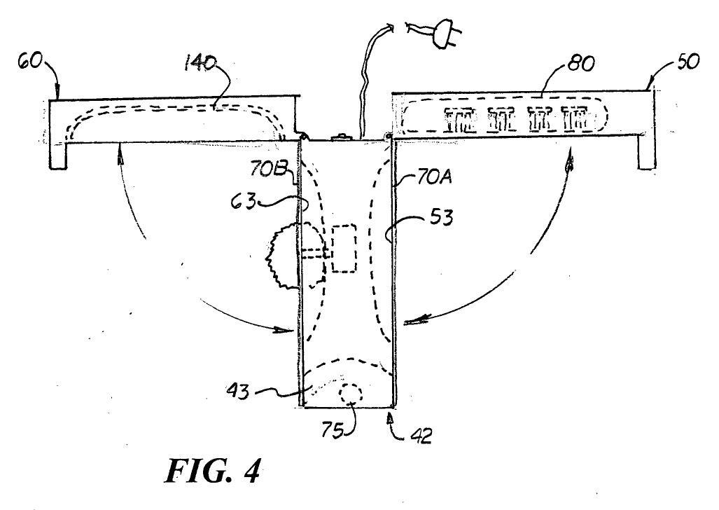

The cleaner 7 is a box structure with a central support member 42 and two pivotally attached doors 50,60, respectfully. The central support member 42 is approximately 4 inches by 4, inches by 9 inches (W×L×H). The two doors 50, 60 are approximately the same width and height and approximately 1½ inches deep. Hinges 62 are disposed between the adjacent edges of the support member 42 and doors 50, 60 that allow them to be selectively opened and closed as shown in FIGS. 1 and 4. Disposed between the perimeter edges 53, 63 of the support member 42 and the two doors 50, 60, respectively, are optional interlocking male and female seals 70A, 70B, respectively, that prevents light from escaping between the adjoining edges 53, 63 of the support member 42 and the doors 50, 60 when the doors are closed. Use of the interlocking seals 70A, 70B is important for preventing light from escaping between the support member 42 and the doors 50, 60 and causing injury during use.

Formed on one end of the support member 42 is an elongated recessed surface 43. Longitudinally aligned and located inside the recessed surface 43 is a germicidal light source 75. In the preferred embodiment, the germicidal light source 75 is an elongated germicidal fluorescent lamp approximately 6 inches in length including the end connectors, and ½ inch in diameter.

Ultraviolet light is divided into three sections according to wavelength. UV-C includes wavelengths from 100 nm to 280 nm, UV-B includes wavelengths from 280 nm to 315 nm, while UV-A includes wavelengths form 315 nm to 400 nm. Only UV-C light is germicidal which deactivates DNA of bacteria and viruses and so destroys their ability to multiply and cause disease. It specifically damages the nucleic acid of organisms by forming covalent bonds to adjacent bases in the DNA. The formation of such bonds prevents the DNA from unzipping during replication and the organisms are unable to reproduce.

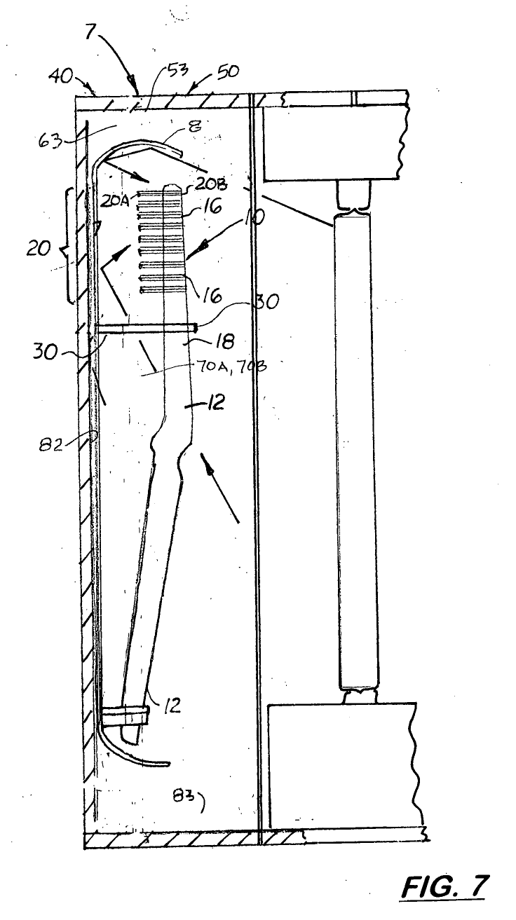

The coherent light from any germicidal lamp 15 rated at 265 nm will have at least 90% of the rays close to 265 nm. Located on the first door 50 and behind the toothbrushes 10 is a reflector 80. The preferred embodiment, the reflector 80 includes an upper concave section 81 that extends inward and surrounds the heads of the toothbrush, a flat intermediate section 82 that extends downward behind the toothbrush handles 12, and a lower section 83 that is perpendicular aligned with the intermediate section 82 that extends inward and under the ends of the handles 12. During operation, the light rays from the germicidal light source 75 are transmitted directly to the proximal ends 20B of the toothbrushes 10 or reflected off the three sections 81-83 of the reflector 80 and towards the distal ends 20A of the bristles 20 as shown in FIG. 7.

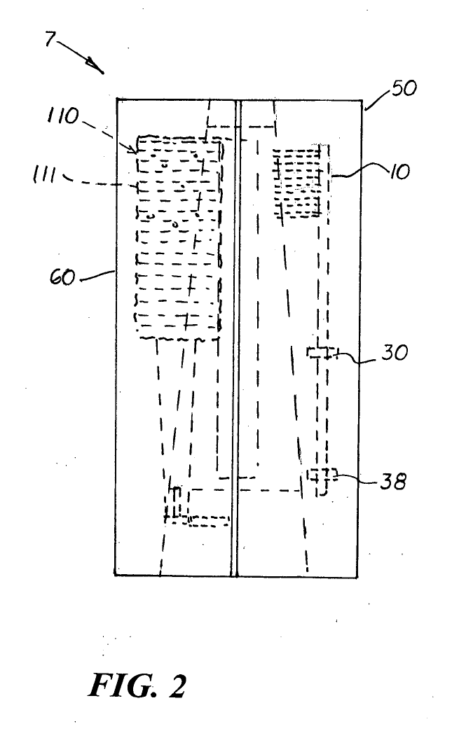

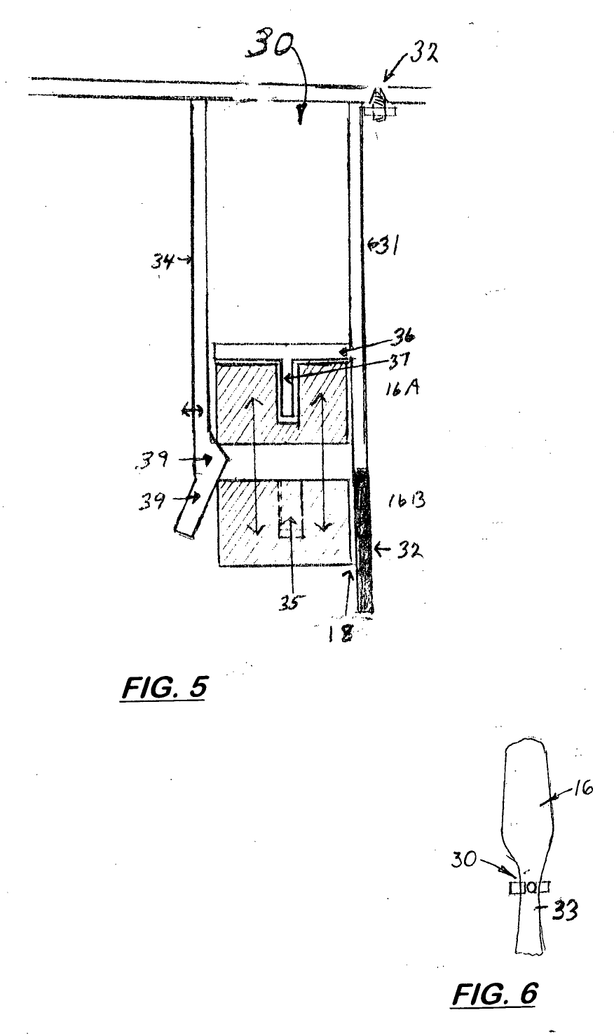

As stated above, four toothbrushes 10 A-D is attached to the inside surface 51 of the first door 50. Each toothbrush 10 A-D is temporarily mounted to the inside surface with two retainer clips 30. In the first embodiment shown in FIGS. 1-2, there are four spring clamp retainers 30 that securely hold four toothbrushes 10 A-D in a longitudinally aligned position in the inside surface 51 of the door 50. FIG. 5 is a detailed top plan view of the spring clamp retainers 30 that allow easy insertion and extraction of a toothbrush 10 and slight rotational adjustment. As shown in FIGS. 5 and 6, each spring clamp retainer 30 includes a fixed arm 31 perpendicular aligned and extending inward from the inside surface 61 of the door 60. A screw 32 is used to securely attach the fixed arm 31 to the inside surface 61. Attached to the distal end of the fixed arm 31 is a short cross member 32. Attached to the cross member 32 is an inward extending peg 37. Formed on each toothbrush head is a small hole 11 designed to receive the peg 37.

Aligned parallel to the fixed arm 31 is a spring arm 34. Attached to the distal end of the spring arm 34 is a diagonally aligned latch 39 that extends over the outer edge of the toothbrush 10 when the toothbrush head 16 is properly position on the retainer 30 and attached to the peg 37 to hold the toothbrush head 16 on the spring clamp retainer 30.

Located below each spring clamp receiver 30 and also attached to the inside surface 51 of the first door 50 is a U-shaped brush holder 38. During use, the brush holder 38 receives the flat distal end of the toothbrush 10. After seating the distal end of the toothbrush 10 into the brush holder 38, the side of the neck of the toothbrush 10 is placed against the extension 32 on the fixed arm 31. The other side of the neck brushes against the sloping edge of the latch 39 on the spring arm 34. Once placed in this position, the toothbrush 10 is then pressed inward so that the peg 37 is inserted into the hole 37. The toothbrush head is then in proper position against the stop 36.

The circular hairbrush 110 is mounted on a vertical axle 120 that extends upward from a lateral short support arm 47 connected to the inside surface of the support member 42. The vertical axle 120 connects to gear box 125 located inside the support member 42. Attached to the opposite end of the gear box 125 is an electrical motor 130. The gear box 125 and motor 130 are designed to rotate the hairbrush 110 approximately 1 revolution every 15 minutes. The electric motor 130 is electrically connected to a main power switch 135 located on the rear surface of the support member 42, which controls operation of the electric motor 130 and the operation of the light source 75. Mounted on the inside surface of the second door 60 is an optional, concave reflector designed to reflect ultraviolet light onto the bristles 111 of the circular hairbrush 110.

An optional timer 85 is mounted on the support member 42 and used to control the length of time the light source 75 is lit. Typically, about 60 seconds is all that is needed to completely sterilize the heads 16 and bristles 20. However, if the toothbrushes 10 or hair brush 110 are wet when placed into the cleaner 7, additional time may be needed. The timer 85 automatically turns off the light source 75 at the set time on the timer 85.

An electrical plug 92 and wires 93, 95 are connected to a 24 volt transformer 94 to provide electrical power to the light source 75. There is also a cutoff switch 99 mounted on the inside surface of each door 50, 60 that prevents the light source 75 from being activated and turns off the light source 75 when the door 50 is open or ajar and not sealed.

As mentioned above, the position of the heads of the toothbrush 10 and hairbrush 110 in relation to the germicidal light source 75 is critical. During use, the location of the light source 75 must be properly positioned on the support member 42 so that light rays from the light source 75 approaches the ends of the bristles 20 at, or greater, than the critical angle in order to achieve total internal reflection in the bristles. This is made possible by adjusting the toothbrushes by means of the screw 32 on the fixed arm or the spring clamp retainer 30 and my adjusting the position of the light source 75 using the screw 78. Typically, the screws 78 are set and fixed in position during mass production.

In compliance with the statute, the invention described herein has been described in language more or less specific as to structural features. It should be understood, however, that the invention is not limited to the specific features shown, since the means and construction shown is comprised only of the preferred embodiments for putting the invention into effect. The invention is therefore claimed in any of its forms or modifications within the legitimate and valid scope of the amended claims, appropriately interpreted in accordance with the doctrine of equivalents.

I claim:

1. (canceled)

2. A germicidal brush cleaner, comprising:
a. a main body;
b. a door attached to said main body;
c. an ultraviolet germicidal light source located inside said main body;
d. an On/Off switch connected to said ultraviolet germicidal light source;
e. at least one toothbrush holder used to hold a toothbrush with bristles each made of a fiber optic said holder able to hold a toothbrush at a position inside said main body so that light rays produced by said ultraviolet germicidal light source may be transmitted directly onto the exposed ends of the bristles and internally reflected in the bristles;
f. a reflector used to reflect light rays from said ultraviolet germicidal light source onto the bristles on a toothbrush placed on said holder;
g. a brush holder used to hold a hairbrush with bristles inside said main body, said brush holder used to hold a hairbrush so that light rays from said ultraviolet germicidal light source are transmitted onto the bristles.

3. The cleaner, as recited in claim 2, wherein said toothbrush holder allows a toothbrush attached thereto to be adjusted in position with respect to said germicidal light source.

4. The cleaner, as recited in claim 2, wherein said germicidal light source is a florescent ultraviolet lamp.

5. The cleaner, as recited in claim 2, wherein said reflector is located around the head of a toothbrush when attached to said toothbrush holder.

6. The cleaner, as recited in claim 2, wherein said germicidal light source is longitudinally aligned in said main body opposite said tooth brush holder.

7. The cleaner, as recited in claim 6, wherein said germicidal light source is located in the corner of said main body opposite said reflector.

8. The cleaner, as recited in claim 2, further including a drain hole formed on said main body to allow water to drain from said main body.

9. The cleaner, as recited in claim 2, further including means for adjusting the position of said germicidal light source in said main body.

10. The cleaner, as recited in claim 2, further including means for adjusting the position of said toothbrush holder in said main body.

11. The cleaner, as recited in claim 2, further including means to determine if said light source is activated when said door is closed.

12. The cleaner, as recited in claim 2, wherein said toothbrush holder automatically aligns a toothbrush inside said main body relative to said light source so that light rays from said light source radiate onto the distal ends of said bristles at a critical angle.

13. Method of sterilizing brushes comprising the following steps:
a. selecting a cleaner with a germicidal light source located therein;
b. selecting a toothbrush with a plurality of bristles each made of a fiber optic attached thereto, said bristles having exposed ends;
c. selecting a hairbrush with a plurality of bristles;
d. placing said toothbrush inside said cleaner so that light from said germicidal light source is transmitted at or near the critical angle of reflection on said exposed ends of said bristles;
e. placing a hairbrush into said cleaner so that light from said germicidal light sources is transmitted onto said bristles; and,
f. activating said germicidal light source so that said toothbrush bristles are sterilized by said germicidal light source.

14. The cleaner, as recited in claim 1, further including an electric motor coupled to said hairbrush holder to rotate said hairbrush.

15. The cleaner, as recited in claim 14, wherein said hairbrush is circular in cross-section.

16. The cleaner, as recited in claim 14, further including a second mirror located inside said main body to reflect ultraviolet light onto the bristles on said hairbrush.

17. The cleaner, as recited in claim 14, further including an ON/OFF switch connected to said electric motor to control activation and deactivation of said electric motor.
